(19) United States
(12) Patent Application Publication
(10) Pub. No.: US 2006/0058703 A1
(43) Pub. Date: Mar. 16, 2006

(54) Optical biopsy instrument

(75) Inventor: Michael Huenerbein, Berlin (DE)

Correspondence Address: ROTHWELL, FIGG, ERNST & MANBECK, P.C., 1425 K STREET, N.W., SUITE 800, WASHINGTON, DC, 20005, US

(73) Assignee: Universitaetsklinikum Charite Medizinische Fakultaet der Humboldt-Universitaet zu Berlin, Berlin (DE)

(21) Appl. No.: 10/538,649

(22) PCT Filed: Dec. 9, 2003
(86) PCT No.: PCT/EP03/13995

§ 371 Date: Jun. 10, 2005

(30) Foreign Application Priority Data
Dec. 10, 2002 (DE) 102 58 483.4

(51) Int. Cl. A61B 10/00

(52) U.S. Cl. 600/567

(57) ABSTRACT

The invention relates to an optical biopsy instrument (100) comprising
(a) a substantially cylindrical cannula (22) with a proximal end (24) and a distal end (26), said cannula (22) having at least one lateral opening (28), and
(b) an endoscope (10) which is axially movable inside the cannula (22).

The biopsy instrument (100) according to the invention permits, for the first time, a targeted removal of tissue samples (34) from narrow duct systems of the body, in particular from milk ducts of the breast, with optimal endoscopic visual monitoring.

The invention relates to an optical biopsy instrument suitable for removing tissue samples from small duct systems, in particular from milk ducts of the breast, and methods for removing tissue samples using the biopsy instrument.

Breast cancer, accounting for approximately 20% of cancer mortality, represents the most common cause of cancer-related death among women in Germany and is therefore a considerable medical and socioeconomic problem. For a favorable prognosis and a successful treatment of breast cancer, early detection of the disease is of the utmost importance. Thus, international studies have confirmed that early detection programmes with (radiological) breast screening can achieve a significant reduction of the mortality rate from breast cancer.

In recent years, however, a significant increase from 4% up to more than 20% has been seen in intraductal carcinomas, that is to say carcinomas of the milk ducts (ductus lactiferi) of the breast (Bässler, Onkologie 4, 1998, 878-895). These tumors of the milk duct system are therefore the present focus of efforts to improve the diagnosis and treatment of carcinoma of the breast. However, early diagnosis of these small tumors is problematic, particularly because of their peculiarity of spreading slowly in the preformed duct system of the breast, and is often insufficient with conventional radiological methods (mammography) or ultrasound of the breast. The clinical symptoms and mammographic findings lead to a diagnosis in only 40% to 60% of patients (Bässler, Onkologie 4, 1998, 878-895).

Endoscopic examination of the milk ducts of the breast, referred to as ductoscopy, could potentially improve the effectiveness of early detection, in particular of intraductal carcinomas. However, it was previously not possible because of the small cross section of the duct. Only the miniaturization of endoscopes has made direct optical examination of these small duct systems possible. In contrast to the situation with other organs, there is only little experience of microendoscopy of the milk ducts hitherto, and these examinations served for locating intraductal papillomas or for other purposes (e.g. Shen et al., Am. Cancer Soc. 89, 2000, 1512-1519; Okazaki et al., Eur. Radiol. 9; 1999; 583-590; Love and Barsky, Lancet 348, 1996, 997-999; Rimbach et al., Zentralbl. Gynakol. 117, 1995, 198-203; Okazaki et al., Jpn. J. Clin. Oncol. 21, 1991, 188-193). Most of these methods, however, do not permit removal of a tissue sample, but at best a collection of epithelial cells by lavage of the duct. Where the possibility of a biopsy is provided for, this is performed blind and not under visual monitoring. However, just the possibility of performing a precise, optically controlled biopsy is absolutely essential for an optimized spreading diagnosis to improve treatment planning and treatment results in respect of breast-preserving therapy of carcinomas of the breast.

Shen et al. (Surg. Endosc. 15, 2001, 1340-1345) describe a biopsy method for milk ducts, wherein a fiber endoscope with a conventional thin biopsy cannula pushed onto it is introduced into the milk duct with visual monitoring, the endoscope is removed, and tissue is aspirated by means of a very thin syringe which is inserted. However, a problem is that the actual biopsy does not take place with visual monitoring as well. For this reason, there is a risk of errors being made, since even the very smallest deviations from the target biopsy site can lead to unreliable diagnoses.

Other publications describe biopsy instruments for other purposes, which likewise do not permit direct endoscopic monitoring of the removal of the sample. U.S. Pat. No. 4,651,753 describes a biopsy instrument intended to be used in connection with an endoscope. The biopsy instrument essentially comprises a cylindrical outer sleeve with an oval lateral opening, a flexible cutting cannula which is arranged coaxially and is axially movable in the outer sleeve and which at its front end carries a cutting blade, and a pusher tube which is arranged coaxially and movably in the cutting cannula. A sample is removed by guiding the instrument through an endoscope to the relevant biopsy site, generating an negative pressure to aspirate a tissue sample through the lateral opening into the inside of the instrument, pushing the cutting cannula forward to cut off the tissue sample, and conveying the tissue sample into a front chamber of the outer cylinder using the pusher tube. A disadvantage of this instrument is its complex structure which runs counter to the goal of miniaturization, as is required for example for use in the milk ducts. Moreover, a direct endoscopic monitoring of the removal of the sample is not enabled. A similar principle, having therefore the same disadvantages, is described in U.S. Pat. No. 5,526,822.

U.S. 01/0047183 A discloses a biopsy instrument comprising a double cannula, in which the two parallel cannula passages (working channel and vacuum channel) are connected to one another via vacuum holes. To remove a tissue sample, tissue is aspirated by vacuum through an opening arranged laterally in the working channel, and the sample is cut off with the aid of a tubular, rotating cutting tube axially movable in the working channel. In the same way as with the instrument described in U.S. Pat. No. 4,651,753, it is again not possible in this case to perform a biopsy with endoscopic monitoring.

It may be stated, in summary, that the clinical use of the promising technique of ductoscopy have hitherto been severely restricted by the poor optical quality, by the lack of a working channel in the available instruments, and in particular by the impossibility of visual monitoring of the removal of the tissue sample. These reasons at present limit the clinical acceptance and widespread application of the method.

The object of the present invention is therefore to make available a biopsy instrument having a simple construction, which permits the removal of tissue samples under direct visual monitoring even in very narrow hollow cavities, in particular in the milk ducts of the breast.

According to the invention, this object is achieved by an optical biopsy instrument having the features of claim 1. The biopsy instrument according to the invention comprises
(a) a substantially cylindrical cannula with a proximal end and a distal end, said cannula having at least one lateral opening, and
(b) an endoscope which is axially movable inside the cannula.

The instrument is therefore distinguished by a very simple structure which for the first time permits direct endoscopic monitoring of tissue removal through the lateral opening of the cannula, even in very fine vessels or duct systems, in particular in the milk ducts of the breast. This therefore avoids the frequent sources of error in biopsies hitherto performed blind. By means of only two essential elements, the cannula and the endoscope, the endoscopy as well as the biopsy is enabled, because the endoscope interacting with the opening allows the performance of all necessary actions, in particular the cutting. Moreover, the simple construction enables a very little dimensioning of the instrument and therefore the accessibility of narrowest duct systems.

Separation of the tissue sample from the rest of the tissue is preferably simplified by providing the at least one lateral opening of the cannula at least in parts with a cutting region on its circumference directed towards the distal end and/or on its circumference directed towards the proximal end. In this way, the tissue sample can be easily separated by suitable movement of the cannula and of the endoscope relative to one another, or only of the cannula. These procedures are explained in detail below.

The cutting region of the lateral opening can advantageously be formed by a ground edge of the circumference of the opening and/or by a toothing of the circumference. A combination of ground edge and toothing is also conceivable. The opening itself can, for example, have a substantially round, oval, elliptic or rectangular configuration.

According to an advantageous embodiment of the invention, an external diameter of the endoscope substantially corresponds to an internal diameter of the cannula or is slightly smaller than this, that is to say a clearance between these components is as small as possible. Such relative dimensioning of the interacting components facilitates, on the one hand, the cutting operation and, on the other hand, the creation of an negative pressure in the cannula chamber, which is additionally facilitated by the fact that the cannula is closed at its distal end by a wall, which is preferably transparent.

The endoscope can alternately be a rigid endoscope or a flexible endoscope, that is to say both mirror endoscopes and glass-fiber endoscopes are applied. However, the rigid endoscope has proven especially advantageous because it is easier to handle during cutting.

Various procedures for removing tissue samples from duct systems using the biopsy instrument according to the invention are conceivable.

According to a first variant:
(a) an optical biopsy instrument comprising
   a substantially cylindrical cannula with a proximal end and a distal end, and with at least one lateral opening, and
   an endoscope which is axially movable inside the cannula, is inserted, with endoscopic monitoring, into the duct as far as the biopsy site,
(b) the tissue sample is brought through the free opening into the inside of the cannula, and
(c) the tissue sample is cut off from the rest of the tissue by pushing the endoscope forward across the lateral opening and/or by pulling the cannula back, until the lateral opening is closed.

According to this first variant, the tissue sample is thus cut off by the cannula and endoscope being displaced counter to one another, so that the tissue sample is cut off by means of a pressure exerted by the endoscope against the cutting region of the opening.

According to a second variant, with the endoscope being pulled back, that is to say with the opening lying free, the cannula is used in the manner of a plane in order to cut off the tissue sample. To do this:
(a) the optical biopsy instrument described above is inserted with endoscopic monitoring into the duct until the lateral opening comes to lie over the biopsy site, and
(b) the tissue sample is brought through the free opening into the inside of the cannula, and
(c) the tissue sample is cut off from the rest of the tissue by moving the cannula, with the lateral opening lying free, together with the fixed endoscope forward or backward and with a light manual pressure being exerted towards the tissue sample.

Further preferred embodiments of the invention will be evident from the other features specified in the dependent claims.

The invention is explained in more detail below on'the basis of an embodiment and with reference to the attached drawings, in which:

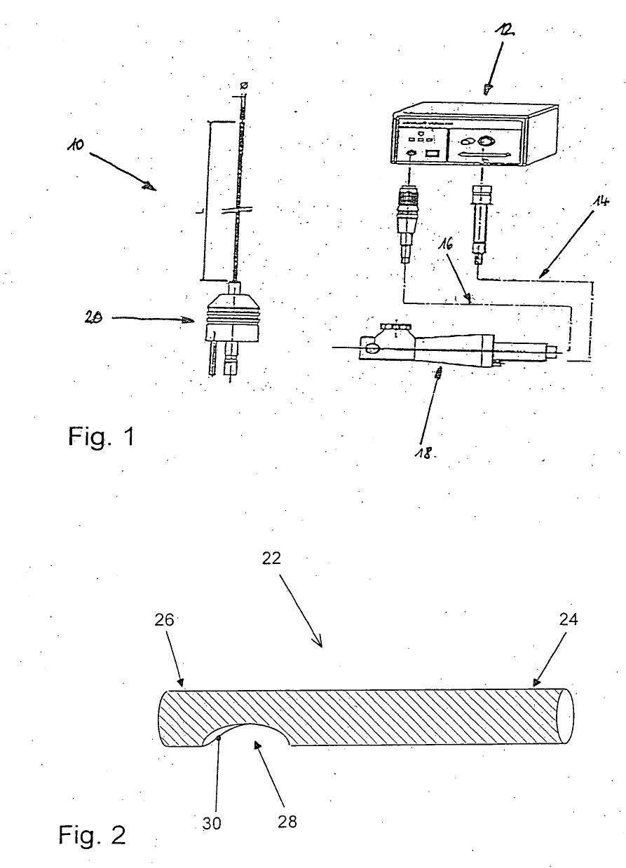

FIG. 1 shows a microendoscopic system consisting of an endoscope, a video handle and a light source,

FIG. 2 shows a perspective side view of a cannula according to the invention,

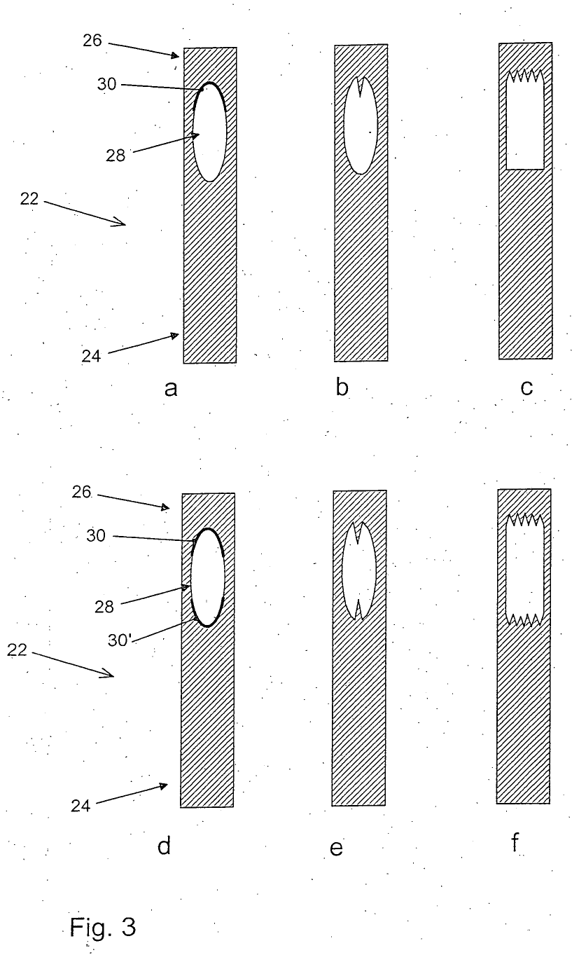

FIGS. 3a to 3f show side views of cannulas according to the invention, with different configurations of the opening and of its cutting regions,

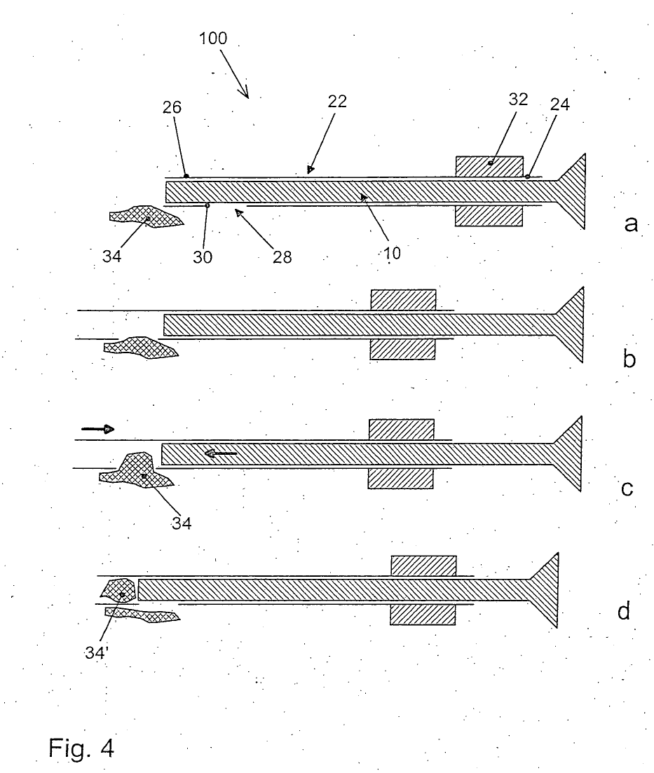

FIGS. 4a to 4d show steps in a method according to the invention for biopsy of a tissue sample according to a first variant, and

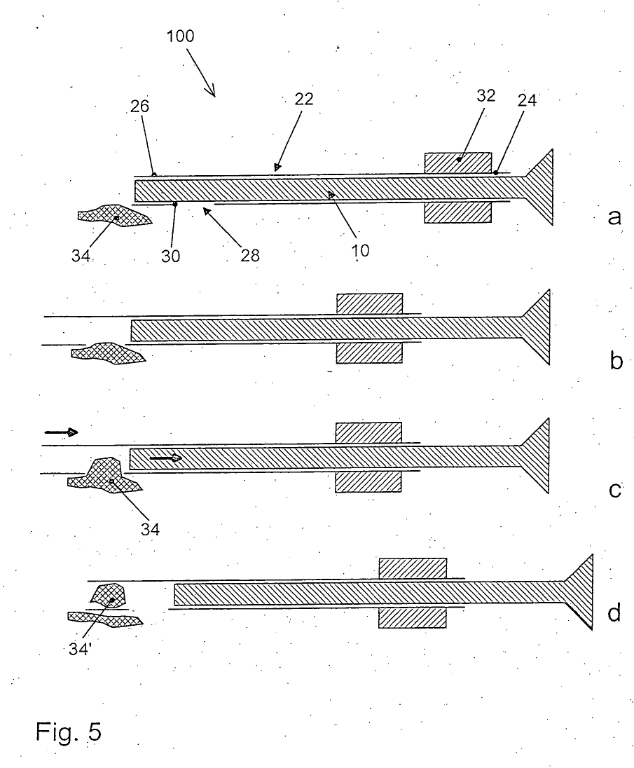

FIGS. 5a to 5d show steps in a method according to the invention for biopsy of a tissue sample according to a second variant.

FIG. 1 is a general view of a microendoscopic system as used in the present invention.

The system comprises an endoscope 10 (ductoscope) which, because of its small size, is suitable for endoscopy of narrow channels, in particular of the milk ducts of the breast (ductus lactiferi). This is a rigid endoscope (from Volpi AG, Switzerland) with an external diameter 0 of 1 mm and a length L of 6 cm. The endoscbpe is based on what is called the gradient index technique (GRIN) and offers a much higher optical resolution and better luminosity compared to fiber-optic endoscopes.

The system further comprises a light source 12, for example a metal halide light source. The light source 12 can be connected to a fiber-optic cable 14 and a camera cable 16, which lead to a video handle 18. The video handle 18 has a camera head (not shown in detail here) which contains a CCD camera with a resolution of 625 lines and a manual focus (2 mm-∞). The CCD camera delivers a digital image signal of excellent quality. The endoscope 10 can be connected to the video handle 18 via a connector head 20 and can thus be connected to the light source 12 via the fiber-optic cable 14 and the camera cable 16.

With its good optical qualities and its easy maneuvering, the system is suitable for wide-ranging clinical use. Although the endoscope has a working channel through which for instance a lavage can be performed, the small diameter does not permit a biopsy. This problem is solved, according to the invention, by using a cannula 22 which can be fitted onto the endoscope 10, as is shown in FIG. 2.

The cannula 22, which essentially has the shape of a hollow cylinder, has a proximal end 24 and a distal end 26. The cannula can be made of a rigid plastic or of metal, for example. Its internal diameter is preferably slightly greater than the external diameter of the endoscope 10, so that there is only slight clearance between endoscope 10 and pushed-on cannula 22. In this specific case, a cannula 22 with an external diameter of 1.2 mm was used. Near the cannula tip, that is to say in the area of its distal end 26, the cannula 22 has a lateral opening 28 which is equipped at least in parts with a cutting region 30.

FIGS. 3a to 3f show a number of embodiments of the cannula 22 which differ in terms of the configuration of the lateral opening 28 and the nature of the cutting region 30. The openings 28 according to Figures a, b, d and e have a substantially oval configuration, whereas the openings 28 according to Figures c and f are largely rectangular. Other configurations of the opening 28 (not shown here), for example with a round or elliptic contour, are also conceivable it is likewise possible for there to be more than one lateral opening 28, for example two openings 28 lying opposite one another.

The three variants (3a to 3c) depicted at the top in the figure have cutting regions 30 only on that area of the opening 28 directed towards the distal end 26 of the cannula 22. By contrast, the three variants (3d to 3f) at the bottom also have cutting regions 30′ on that area of the opening 28 directed towards the proximal end 24 of the cannula 22. It is likewise conceivable to have a cutting region extending about the entire circumference of the opening 28, or, for certain maneuvers of the biopsy instrument, only cutting regions 30′ on the proximal side.

In the variants 3a and 3d, the cutting regions 30, 30′ are in the form of a ground edge of the cannula 22 (indicated by bold lines). On the other hand, the cutting regions 30, 30′ of the other variants consist of a toothing, the cutting regions 30, 30′ of variants 3b and 3e each being in the form of an individual tooth, and those of variants 3c and 3f being in the form of a finer multiple toothing. In addition, it may also be advantageous to combine ground edge and toothing with one another, for instance by way of a toothed ground edge or by way of a toothing on one side of the opening and a ground edge on the other side.

Two different methods of performing a biopsy of a tissue sample using the biopsy instrument according to the invention are shown diagrammatically in FIGS. 4 and 5.

According to the first method outlined in FIG. 4, the biopsy instrument, designated overall by 100 and consisting of the endoscope 10 and of the cannula 22 pushed onto the latter, is introduced with endoscopic monitoring into a hollow vessel to be examined (not shown), in particular into a milk duct (FIG. 4a). In this phase, the endoscope 10 is preferably situated in a maximally advanced position inside the cannula 22, so that the distal cannula tip is essentially flush with the tip of the endoscope 10. For better handling, the cannula 22 has a grip 32 at its proximal area 24. As soon as a tissue sample 34 to be removed comes into the viewing area of the endoscope 10, the insertion movement of the instrument 100 is ended.

A hand grip is then used to push the cannula 22 forward until the opening 28 of the cannula 22 is freed, that is to say is no longer closed by the endoscope 10 lying within, and the opening 28 comes to lie over the tissue sample 34 (FIG. 4b).

In the next step, the tissue sample 34 to be removed is brought through the opening 28 into the inside of the cannula 22 (FIG. 4c). On the one hand, this can happen spontaneously as a result of a tissue stress acting against the cannula 22 and the opening 28. Optionally, the inward movement of the tissue can be assisted by a slight negative pressure generated in the cannula 22, which negative pressure can be generated by the preceding advance movement of the cannula 22. In this case, the distal cannula tip can be closed off by a transparent wall (not shown). Optionally, the negative pressure can also be generated via the working channel present in the endoscope 10.

As soon as the tissue sample 34 has moved into the cannula, it is separated from the rest of the tissue by means of the endoscope 10 being pushed forward or the cannula 22 being retracted. It is also advantageously possible to combine both movements with one another, by moving cannula 22 and endoscope 10 in opposite directions (see arrows in FIG. 4c). Because of the shearing action of the cutting region 30 of the opening 28 of the cannula 22, the tissue is clamped and cut off, the separated tissue sample 34′ being pushed into the inside of the distal area 26 of the cannula 22 (FIG. 4d). The entire biopsy instrument 100 together with the tissue sample 34′ can then be removed from the duct system. For this purpose too, the cannula tip can also advantageously be closed off by a window in order to prevent loss of the tissue sample 34′ as the instrument 100 is being withdrawn.

A second variant of the method for performing a biopsy of a tissue sample is shown in FIG. 5. Insertion of the biopsy instrument 100 (FIG. 5a) and introduction of the tissue sample 34 (FIGS. 5b and 5C) take place analogously to the method described above in FIG. 4. The difference from the latter lies in the way in which the tissue sample 34 is separated, which is done by using the cannula 22 in the manner of plane. As soon as the tissue sample 34 has invaded into the cannula 22, the whole instrument 100, consisting of the cannula 22 and of the endoscope 10 fixed in it, is moved in a common direction (see arrows in FIG. 5c) with a slight manual pressure being exerted against the tissue sample 34, so that the cutting region 30 of the cannula 22 engages the tissue sample 34 and separates it (FIG. 5d). Depending on whether the cutting region 30 is situated at the distal or the proximal side of the opening 28, the unidirectional movement of separation can take place in one direction or the other. In the example shown, where the cutting region 30 is arranged on the distal side of the opening 28, the tissue separation is done by pulling the instrument 100 back.

In a further procedure (not shown here) for performing a biopsy, a second lateral opening of the cannula 22 is advantageous. With the endoscope 10 pulled back, the cannula 22 is pressed with a levering movement onto the tissue sample 34 to be removed, in such a way that the tissue passes through the first lateral opening 28 into the inside of the cannula and then passes in parts through the opposite second opening out of the cannula 22 back into the duct. The separation of the sample 34 is then effected either by once again moving endoscope 10 and cannula 22 counter to one another, as in FIG. 4, or by using the instrument 100 like a lever, as in FIG. 5. A cutting region 30 on the second lateral opening is not absolutely necessary for this variant.

The entire process of removing a sample in accordance with all the methods described can be monitored and controlled under optimal endoscopic viewing. In particular, the removal site can be very precisely targeted by means of the biopsy instrument 100 according to the invention. Incorrect samples, which were previously common in “blind” biopsies as a result of slightly missing the removal site, are now avoided.

Given the high medical and socio-economic relevance of breast cancer, it is assumed that the biopsy instrument according to the invention and the methods for its use will gain widespread acceptance among doctors and patients. The method is simple and inexpensive and can be used extensively after a short period of training of doctors. Since the biopsy instrument is a low-cost product, it can be used both in hospitals and also in general practice.

### Reference Numbers

10 endoscope
12 light source
14 fiber-optic cable
16 camera cable
18 video handle with camera head
20 connector head
22 cannula
24 proximal end
26 distal end
28 opening
30 cutting region
32 grip
34 tissue sample

1. Optical biopsy instrument (100), comprising
(a) a substantially cylindrical cannula (22) with a proximal end (24) and a distal end (26), said cannula (22) having at least one lateral opening (28), and
(b) an endoscope (10) which is axially movable inside the cannula (22).

2. Optical biopsy instrument (100) according to claim 1, characterized in that the at least one lateral opening (28) of the cannula (22) has at least in parts a cutting region (30) at its area being directed towards the distal end (26) and/or at its area being directed towards the proximal end (24).

3. Optical biopsy instrument (100) according to claim 2, characterized in that the cutting region (30) is formed by a ground edge of the circumference of the at least one lateral opening (28) or by a toothing of the circumference or by both measures concurrently.

4. Optical biopsy instrument (100) according to claim 1, characterized in that the at least one lateral opening (28) has a substantially round, oval, elliptic or rectangular configuration.

5. Optical biopsy instrument (100) according to claim 1, characterized in that the cannula (22) is closed at its distal end (26) by a wall, in particular by a transparent wall.

6. Optical biopsy instrument (100) according to claim 1, characterized in that an external diameter of the endoscope (10) substantially corresponds to an internal diameter of the cannula (22) or is slightly smaller than this.

7. Optical biopsy instrument (100) according to claim 1, characterized in that an external diameter of the cannula is 1.2 mm at most.

8. Optical biopsy instrument (100) according to claim 1, characterized in that the endoscope (10) is a rigid endoscope or a flexible glass-fibre endoscope.

9. Use of an optical biopsy instrument (100) according to claim 1 for endoscopy and/or biopsy of duct systems having small diameters, in particular of milk ducts of mammary glands.

10. Method for sampling tissue samples in duct systems, wherein
(a) an optical biopsy instrument (100), comprising
a substantially cylindrical cannula (22) with a proximal end (24) and a distal end (26), said cannula (22) having at least one lateral opening (28), and
an endoscope (10) which is axially movable inside the cannula (22) is introduced, under endoscopic monitoring, into the duct up to a biopsy site,
(b) the tissue sample (34) is brought through the free lying opening (28) into an interior of the cannula (22), and
(c) the tissue sample (34) is separated from the rest of the tissue by moving forward the endoscope (10) across the lateral opening (28) and/or by retracting the endoscope (10), until the lateral opening (28) is closed.

11. Method for sampling tissue samples in duct systems, wherein
(a) an optical biopsy instrument (100), comprising
a substantially cylindrical cannula (22) with a proximal end (24) and a distal end (26), said cannula (22) having at least one lateral opening (28), and
an endoscope (10) which is axially movable inside the cannula (22) is introduced, under endoscopic monitoring, into the duct, until the lateral opening (28) comes to lie over a biopsy site,
(b) the tissue sample (34) is brought through the free lying opening (28) into an interior of the cannula (22), and
(c) the tissue sample (34) is separated from the rest of the tissue by moving the cannula (22) having a free lying lateral opening (28) together with the fixed endoscope (10) forward or backward, thereby manually exerting a gentle pressure against the tissue sample (34).
